(19) United States
(12) Patent Application Publication
(10) Pub. No.: US 2016/0158325 A1
(43) Pub. Date: Jun. 9, 2016

(54) METHOD OF TREATING FIBROPROLIFERATIVE DISORDERS INCLUDING DUPUYTREN'S DISEASE WITH ONE OR MORE SPECIFIC HUMAN MATRIX METALLOPROTEINASE AND A TNF ANTAGONIST

(71) Applicant: Glenn LARSEN, Sudbury, MA (US)

(72) Inventor: Glenn Larsen, Sudbury, MA (US)

(73) Assignee: 180 Therapeutics LP, Sudbury, MA (US)

(21) Appl. No.: 14/904,391

(22) PCT Filed: Jul. 9, 2014
(86) PCT No.: PCT/US14/45988

§ 371 Date: Jan. 11, 2016

Related U.S. Application Data
(60) Provisional application No. 61/845,366, filed on Jul. 11, 2013.

(51) Int. Cl. A61K 38/48; A61K 9/00; A61K 45/06

(52) U.S. Cl. CPC A61K 38/4886; A61K 45/06; A61K 9/0014; C12Y 304/24007

(57) ABSTRACT

The subject invention also provides a method of treating a subject afflicted with a fibroproliferative disorder comprising periodically administering to the patient an amount of one or more human matrix metalloproteinase, wherein the one or more human matrix metalloproteinase are selected from human metalloproteinase-1 (MMP-1), human metalloproteinase-2 (MMP-2), human metalloproteinase-3 (MMP-3), human metalloproteinase-7 (MMP-7), human metalloproteinase-8 (MMP-8), human metalloproteinase-9 (MMP-9), human metalloproteinase-10 (MMP-10), human metalloproteinase-11 (MMP-11), metalloproteinase-12 (MMP-12), and human metalloproteinase-13 (MMP-13), and wherein the amount is effective to treat the subject. In an embodiment, the invention further comprises periodically administering to the subject an amount of TNF antagonist, wherein the amount of one or more the human matrix metalloproteinase and the amount of TNF antagonist when taken together are effective to treat the subject.

This application claims priority of U.S. Provisional Application No. 61/845,366, filed Jul. 11, 2013, the entire contents of which are hereby incorporated by reference herein.

Throughout this application, various publications are referred to by first author and year of publication. Full citations for these publications are presented in a References section immediately before the claims. Disclosures of the publications cited in the References section are hereby incorporated by reference in their entireties into this application in order to more fully describe the state of the art as of the date of the invention described herein.

### BACKGROUND OF THE INVENTION

Dupuytren's disease, alternatively known as palmar fibromatosis (or in its established disease state Dupuytren's contracture), is a disease associated with the buildup of extracellular matrix materials such as collagen on the connective tissue of the hand (the palmar fascia) causing it to thicken and shorten with the physical effect of causing the fingers to curl, most commonly the ring finger and little finger.

Dupuytren's disease affects approximately 5% of the white Caucasian population. The commonest manifestation is progressive flexion contracture of the digits of the hand, resulting in significantly compromised function. It affects both males and females, but the incidence is higher in males.

The causes of Dupuytren's disease are not well understood and underlying disease is not currently curable.

Treatment of Dupuytren's disease has traditionally been invasive surgical techniques. Primarily, the treatment has involved surgical excision of the offending tissue. In severe or recurrent disease, the surgical excision may be combined with excision of the overlying palmar skin and resurfacing of the cutaneous defect with full-thickness skin graft. Surgery is typically followed by prolonged rehabilitation, usually lasting 3 months and complications have been reported in up to 20% of cases. Such surgical correction is the mainstay treatment of later stage disease when secondary changes to tendons and joints have developed. A less invasive surgical intervention is needle fasciotomy in which the fibrous bands (contractures) in connective tissue are divided using the bevel of a needle.

To date collagenase therapies have appeared relatively effective (however with a high degree of side effects) in treatment of contracture of the metacarpophalangel joint, whilst the correction of proximal interphalangeal joints has been much less satisfactory. Furthermore, as with surgical interventions, recurrence can be expected, but in the case of early collagenase trials, which involve enzymatically cutting the cord, recurrence is high, especially for disease affecting the proximal interphalangeal joint.

Other non-surgical treatments that have been proposed include application of vitamin E cream applied as topical therapy, ltrasonic therapy and low-dose radiation therapy (for slowing the progression of early stage disease), such as X-rays and electron beam therapy.

Most research for treatments of Dupuytren's disease has focused on detecting pre-disposition to Dupuytren's (e.g. US-A-2004/0161761) and on the extracellular matrices produced, which has resulted in the collagenase-based treatments. There has been very little conclusive insight into potential treatments gained from studies into the biochemical pathway of Dupuytren's disease.

There remains a need for novel therapeutic intervention in the treatment and/or prevention of (e.g. progression of) Dupuytren's disease and other musculoskeletal fibroproliferative disorders.

### Combination Therapy

The administration of two drugs to treat a given condition, such as a fibroproliferative disorder, raises a number of potential problems. In vivo interactions between two drugs are complex. The effects of any single drug are related to its absorption, distribution, and elimination. When two drugs are introduced into the body, each drug can affect the absorption, distribution, and elimination of the other and hence, alter the effects of the other. For instance, one drug may inhibit, activate or induce the production of enzymes involved in a metabolic route of elimination of the other drug (Guidance for Industry, 1999). In one example, combined administration of GA and interferon (IFN) has been experimentally shown to abrogate the clinical effectiveness of either therapy. (Brod 2000) In another experiment, it was reported that the addition of prednisone in combination therapy with IFN-β antagonized its up-regulator effect. Thus, when two drugs are administered to treat the same condition, it is unpredictable whether each will complement, have no effect on, or interfere with, the therapeutic activity of the other in a human subject.

Not only may the interaction between two drugs affect the intended therapeutic activity of each drug, but the interaction may increase the levels of toxic metabolites (Guidance for Industry, 1999). The interaction may also heighten or lessen the side effects of each drug. Hence, upon administration of two drugs to treat a disease, it is unpredictable what change will occur in the negative side profile of each drug. In one example, the combination of natalizumab and interferon was observed to increase the risk of unanticipated side effects. (Vollmer, 2008; Rudick 2006; Kleinschmidt-DeMasters, 2005; Langer-Gould 2005)

Additionally, it is difficult to accurately predict when the effects of the interaction between the two drugs will become manifest. For example, metabolic interactions between drugs may become apparent upon the initial administration of the second drug, after the two have reached a steady-state concentration or upon discontinuation of one of the drugs (Guidance for Industry, 1999).

Therefore, the state of the art at the time of filing is that the effects of a combination therapy of two drugs, in particular human matrix metalloproteinase and TNF antagonist, cannot be predicted until experimental results are available.

### BRIEF SUMMARY OF THE INVENTION

The subject invention provides a method of treating a subject afflicted with a fibroproliferative disorder comprising periodically administering to the patient an amount of one or more human matrix metalloproteinase, wherein the one or more human matrix metalloproteinase are selected from human metalloproteinase-1 (MMP-1), human metalloproteinase-2 (MMP-2), human metalloproteinase-3 (MMP-3), human metalloproteinase-7 (MMP-7), human metalloproteinase-8 (MMP-8), human metalloproteinase-9 (MMP-9), human metalloproteinase-10 (MMP-10), human metalloproteinase-11 MMP-11), metalloproteinase-12 (MMP-12), and human metalloproteinase-13 (MMP-13), and wherein the amount is effective to treat the subject.

The subject invention also provides a method of treating a subject afflicted with a fibroproliferative disorder comprising periodically administering to the patient an amount of one or more human matrix metalloproteinase, wherein the one or more human matrix metalloproteinase are selected from human metalloproteinase-1 (MMP-1), human metalloproteinase-2 (MMP-2), human metalloproteinase-3 (MMP-3), human metalloproteinase-7 (MMP-7), human metalloproteinase-8 (MMP-8), human metalloproteinase-9 (MMP-9), human metalloproteinase-10 (MMP-10), human metalloproteinase-11 (MMP-11), metalloproteinase-12 (MMP-12), and human metalloproteinase-13 (MMP-13), and wherein the amount is effective to treat the subject, further comprising periodically administering to the subject an amount of TNF antagonist, wherein the amount of one or more the human matrix metalloproteinase and the amount of TNF antagonist when taken together are effective to treat the subject.

The subject invention also provides a package comprising:
a) a first pharmaceutical composition comprising an amount of human matrix metalloproteinase and a pharmaceutically acceptable carrier, wherein the human matrix metalloproteinase is selected from human metalloproteinase-1 (MMP-1), human metalloproteinase-2 (MMP-2), human metalloproteinase-3 (MMP-3), human metalloproteinase-7 (MMP-7), human metalloproteinase-8 (MMP-8), human metalloproteinase-9 (MMP-9), human metalloproteinase-10 (MMP-10), human metalloproteinase-11 (MMP-11), metalloproteinase-12 (MMP-12), and human metalloproteinase-13 (MMP-13);
b) a second pharmaceutical composition comprising an amount of TNF antagonist and a pharmaceutically acceptable carrier; and
c) instructions for use of the first and second pharmaceutical compositions together to treat a subject afflicted with a fibroproliferative disorder.

The subject invention also provides a kit for use in treating a subject afflicted with a fibroproliferative disorder comprising:
i. a syringe containing one or more human matrix metalloproteinase wherein the one or more human matrix metalloproteinase is selected from human metalloproteinase-1 (MMP-1), human metalloproteinase-2 (MMP-2), human metalloproteinase-3 (MMP-3), human metalloproteinase-7 (MMP-7), human metalloproteinase-8 (MMP-8), human metalloproteinase-9 (MMP-9), human metalloproteinase-10 (MMP-10), human metalloproteinase-11 (MMP-11), metalloproteinase-12 (MMP-12), and human metalloproteinase-13 (MMP-13); and/or
ii. a syringe containing a TNF antagonist.

The subject invention also provides a method of treating a human patient afflicted with a fibroproliferative disorder comprising periodically administering to the patient an amount of a bi-specific antibody comprising a human matrix metalloproteinase specificity and a TNF antagonist specificity.

The subject invention also provides for the use of one or more human matrix metalloproteinase, wherein the one or more human matrix metalloproteinase are selected from human metalloproteinase-1 (MMP-1), human metalloproteinase-2 (MMP-2), human metalloproteinase-3 (MMP-3), human metalloproteinase-7 (MMP-7), human metalloproteinase-8 (MMP-8), human metalloproteinase-9 (MMP-9), human metalloproteinase-10 (MMP-10), human metalloproteinase-11 (MMP-11), metalloproteinase-12 (MMP-12), and human metalloproteinase-13 (MMP-13) in the manufacture of a medicament for treating a subject suffering from a fibroproliferative disorder.

The subject invention also provides for the use of one or more human matrix metalloproteinase, wherein the one or more human matrix metalloproteinase are selected from human metalloproteinase-1 (MMP-1), human metalloproteinase-2 (MMP-2), human metalloproteinase-3 (MMP-3), human metalloproteinase-7 (MMP-7), human metalloproteinase-8 (MMP-8), human metalloproteinase-9 (MMP-9), human metalloproteinase-10 (MMP-10), human metalloproteinase-11 (MMP-11), metalloproteinase-12 (MMP-12), and human metalloproteinase-13 (MMP-13) and a TNF antagonist in the manufacture of a medicament for treating a subject suffering from a, fibroproliferative disorder.

The subject invention also provides for a pharmaceutical composition comprising one or more human matrix metalloproteinase and a TNF antagonist for use in treating a subject suffering from a fibroproliferative disorder.

### BRIEF DESCRIPTION OF THE SEVERAL VIEWS OF THE DRAWINGS

Not Applicable

### DETAILED DESCRIPTION OF THE INVENTION

The compositions and methods of the present invention enable progression of Dupuytren's (and other fibromatosis and like disease) to be slowed or halted, and even reversed. It has particular advantages in that treatment of the early disease state of Dupuytren's (and other fibromatosis and like disease) can be prevented from progressing to an established later state disease and avoid surgical intervention and the associated recovery time.

Compositions and methods of the present invention enable the treatment, prevention and inhibition of progression and even reversal of musculoskeletal adhesions such as adhesive capsulitis and tendon adhesion (such as adhesion of the proximal interphalangeal joint in established disease state Dupuytren's disease).

The subject invention provides a method of treating a subject afflicted with a fibroproliferative disorder comprising periodically administering to the patient an amount of one or more human matrix metalloproteinase, wherein the one or more human matrix metalloproteinase are selected from human metalloproteinase-1 (MMP-1), human metalloproteinase-2 (MMP-2), human metalloproteinase-3 (MMP-3), human metalloproteinase-7 (MMP-7), human metalloproteinase-8 (MMP-8), human metalloproteinase-9 (MMP-9), human metalloproteinase-10 (MMP-10), human metalloproteinase-11 (MMP-11), metalloproteinase-12 (MMP-12), and human metalloproteinase-13 (MMP-13), and wherein the amount is effective to treat the subject.

In one embodiment, the method further comprising periodically administering to the subject an amount of TNF antagonist, wherein the amount of one or more the human matrix metalloproteinase and the amount of TNF antagonist when taken together are effective to treat the subject.

In one embodiment, the amount of one or more human matrix metalloproteinase and the amount of TNF antagonist are administered adjunctively and/or concomitantly.

In one embodiment, the fibroproliferative disorder is a fibromatosis disease. In another embodiment, the fibroproliferative disorder is selected from Dupuytren's disease, plantar fibromatosis, adhesive capsulitis and Peyronie's disease. In another embodiment the fibroproliferative disorder is Dupuytren's disease.

In one embodiment, the one or more human matrix metalloproteinase is human metalloproteinase-1 (MMP-1).

In one embodiment, the method is for the treatment of early disease state fibroproliferative disorder.

In one embodiment, the method is also for the treatment of established disease state fibroproliferative disorder.

In one embodiment, the amount of one or more human matrix metalloproteinase and/or the amount of TNF antagonist are administered or are formulated for injection directly into diseased tissue. In another embodiment, the amount of one or more human matrix metalloproteinase and/or the amount of TNF antagonist are administered or are formulated for topical application. In another embodiment, the amount of one or more human matrix metalloproteinase and/or the amount of TNF antagonist are administered or are formulated for intravenous therapy.

In one embodiment, the TNF antagonist is selected from one or more of Infliximab, Adalimumab, Certolizumab pegol, Golimumab or Etanercept.

In one embodiment, an amount of a therapeutic, prophylactic or progression-inhibiting DAMP antagonist and/or an AGE inhibitor is also administered to the human patient.

In one embodiment, an amount of a therapeutic, prophylactic or progression-inhibiting Alarmin antagonist and/or an AGE inhibitor is also administered to the human patient.

In one embodiment the Alarmin antagonist is one or more of antagonist of HMGB1, S100A8, S100A9, SI00A8/9, S100A12.

In one embodiment, the amount of one or more of the human matrix metalloproteinase and/or the amount of TNF antagonist is administered once daily. In another embodiment, the amount of one or more of the human matrix metalloproteinase and/or the TNF antagonist is administered weekly, biweekly, monthly or bimonthly.

In one embodiment, the amount of one or more of the human matrix metalloproteinase is between 0.01 and 10 mg. In another embodiment, the amount of one or more of the human matrix metalloproteinase is between 0.01 and 2 mg.

In one embodiment, the amount of TNF antagonist is between 0.05-5.0 times the clinical dose of TNF antagonist administered for Rheumatoid Arthritis. In another embodiment the amount of TNF antagonist is between 0.1-3.0 times the clinical dose of THF antagonist administered for Rheumatoid Arthritis. In another embodiment the clinical dose of THF antagonist administered for Rheumatoid Arthritis is 100 mg and therefore the amount of TNF antagonist is between 10 mg and 300 mg.

In one embodiment, the amount of one or more of the human matrix metalloproteinase and the amount of TNF antagonist when taken together is effective to alleviate a symptom of a fibroproliferative disorder in the subject.

In one embodiment, the amount of one or more of the human matrix metalloproteinase and the amount of TNF antagonist when taken together is effective to improve the subject's quality of life.

In one embodiment, the amount of one or more of the human matrix metalloproteinase and the amount of TNF antagonist when taken together is effective to improve the general health status of the subject.

In one embodiment, in the administration of one or more of the human matrix metalloproteinase substantially precedes the administration of TNF antagonist.

In one embodiment, the subject is receiving human matrix metalloproteinase therapy of one or more human matrix metalloproteinase prior to initiating TNF antagonist therapy.

In one embodiment, the administration of TNF antagonist substantially precedes the administration of human matrix metalloproteinase.

In one embodiment, the subject is receiving TNF antagonist therapy prior to initiating human matrix metalloproteinase therapy of one or more human matrix metalloproteinase.

In one embodiment, it comprising administration of an amount of a therapeutic, prophylactic or progression-inhibiting DAMP antagonist and/or an AGE inhibitor is also administered to the human patient.

In one embodiment, the DAMP antagonist is an Alarmin antagonist.

In one embodiment, the Alarmin antagonist is one or more of antagonist of HMGB1, S100A8, S100A9, SI00A8/9, S100A12.

In one embodiment, each of the amount of one or more human matrix metalloproteinase when taken alone, and the amount of TNF antagonist when taken alone is effective to treat the subject.

In one embodiment, either the amount of one or more human matrix metalloproteinase when taken alone, or the amount of TNF antagonist when taken alone, or each such amount when taken alone is not effective to treat the subject.

In one embodiment, the subject is a human.

In one embodiment, the progression of the fibroproliferative disorder is reduced or prevented.

In one embodiment, there is a regression of the fibroproliferative disorder.

In one embodiment, hypersensitivity or allergic reactions during the treatment of the subject is reduced.

In one embodiment, the amount of one or more human matrix metalloproteinase is administered 0 minutes to 48 hours after the TNF antagonist is administered. In another embodiment, the amount of the TNF antagonist is administered within 48 hours after the amount of one or more human matrix metalloproteinase is administered.

In one embodiment, the amount of the TNF antagonist is administered approximately 24 hours after the amount of one or more human matrix metalloproteinase is administered. In another embodiment the amount of the TNF antagonist is administered approximately 15 to 30 minutes after the amount of one or more human matrix metalloproteinase is administered. In another embodiment the amount of one or more human matrix metalloproteinase is administered within 48 hours after the TNF antagonist is administered.

In one embodiment the amount of one or more human matrix metalloproteinase is administered approximately 24 hours after the TNF antagonist is administered. In another embodiment the amount of one or more human matrix metalloproteinase is administered approximately 1 to 60 minutes after the TNF antagonist is administered.

The subject invention also provides a method of treating a subject afflicted with a fibroproliferative disorder comprising periodically administering to the patient an amount of one or more human matrix metalloproteinase, wherein the one or more human matrix metalloproteinase are selected from human metalloproteinase-1 (MMP-1), human metalloproteinase-2 (MMP-2), human metalloproteinase-3 (MMP-3), human metalloproteinase-7 (MMP-7), human metalloproteinase-8 (MMP-8), human metalloproteinase-9 (MMP-9), human metalloproteinase-10 (MMP-10), human metalloproteinase-11 (MMP-11), metalloproteinase-12 (MMP-12), and human metalloproteinase-13 (MMP-13), and wherein the amount is effective to treat the subject, further comprising periodically administering to the subject an amount of TNF antagonist, wherein the amount of one or more the human matrix metalloproteinase and the amount of TNF antagonist when taken together are effective to treat the subject.

The subject invention also provides a package comprising:
a) a first pharmaceutical composition comprising an amount of human matrix metalloproteinase and a pharmaceutically acceptable carrier, wherein the human matrix metalloproteinase is selected from human metalloproteinase-1 (MMP-1), human metalloproteinase-2 (MMP-2), human metalloproteinase-3 (MMP-3), human metalloproteinase-7 (MMP-7), human metalloproteinase-8 (MMP-8), human metalloproteinase-9 (MMP-9), human metalloproteinase-10 (MMP-10), human metalloproteinase-11 (MMP-11), metalloproteinase-12 (MMP-12), and human metalloproteinase-13 (MMP-13);
b) a second pharmaceutical composition comprising an amount of TNF antagonist and a pharmaceutically acceptable carrier; and
c) instructions for use of the first and second pharmaceutical compositions together to treat a subject afflicted with a fibroproliferative disorder.

In one embodiment, the fibroproliferative disorder is Dupuytren's disease.

In one embodiment, the first pharmaceutical composition and/or the second pharmaceutical composition is contained within a syringe for injection into a subject.

The subject invention also provides a kit for use in treating a subject afflicted with a fibroproliferative disorder comprising:
i. a syringe or vial containing one or more human matrix metalloproteinase wherein the one or more human matrix metalloproteinase is selected from human metalloproteinase-1 (MMP-1), human metalloproteinase-2 (MMP-2), human metalloproteinase-3 (MNP-3), human metalloproteinase-7 (MMP-7), human metalloproteinase-8 (MMP-8), human metalloproteinase-9 (MMP-9), human metalloproteinase-10 (MMP-10), human metalloproteinase-11 (MMP-11), metalloproteinase-12 (MMP-12), and human metalloproteinase-13 (MMP-13); and/or
ii. a syringe or vial containing a TNF antagonist

The subject invention also provides a method of treating a human patient afflicted with a fibroproliferative disorder comprising periodically administering to the patient an amount of a bi-specific antibody comprising a human matrix metalloproteinase specificity and a TNF antagonist specificity.

The subject invention also provides for the use of one or more human matrix metalloproteinase, wherein the one or more human matrix metalloproteinase are selected from human metalloproteinase-1 (MMP-1), human metalloproteinase-2 (MMP-2), human metalloproteinase-3 (MMP-3), human metalloproteinase-7 (MMP-7), human metalloproteinase-8 (MMP-8), human metalloproteinase-9 (MMP-9), human metalloproteinase-10 (MMP-10), human metalloproteinase-11 (MMP-11), metalloproteinase-12 (MMP-12), and human metalloproteinase-13 (MMP-13) in the manufacture of a medicament for treating subject suffering from a fibroproliferative disorder.

The subject invention also provides for the use of one or more human matrix metalloproteinase, wherein the one or more human matrix metalloproteinase are selected from human metalloproteinase-1 (MMP-1), human metalloproteinase-2 (MMP-2), human metalloproteinase-3 (MMP-3), human metalloproteinase-7 (MMP-7), human metalloproteinase-8 (MMP-8), human metalloproteinase-9 (MMP-9), human metalloproteinase-10 (MMP-10), human metalloproteinase-11 (MMP-11), metalloproteinase-12 (MMP-12), and human metalloproteinase-13 (MMP-13) and a TNF antagonist in the manufacture of a medicament for treating a subject suffering from a fibroproliferative disorder.

The subject invention also provides for a pharmaceutical composition comprising one or more human matrix metalloproteinase and a TNF antagonist for use in treating a subject suffering from a fibroproliferative disorder.

For the foregoing embodiments, each embodiment disclosed herein is contemplated as being applicable to each of the other disclosed embodiments. In addition, the elements recited in the packaging and pharmaceutical composition embodiments can be used in the method and use embodiments described herein.

A benefit of this invention is the reduction of—hypersensitivity to active drug product. The invention increases the safety and reduces the allergic reaction in subjection, especially when compared to other treatments of Dupuytren's disease. For example, in the clinical trials for Xiapex, which is a Collagenase clostridium histolyticum used as an injectable treatment for Dupuytren's contracture, it was found that up to 17% of the subjects had allergic reactions. The present invention reduces this percentage of subjects who have allergic reactions and other drug related adverse side effects.

### Pharmaceutically Acceptable Salts

The active compounds for use according to the invention may be provided in any form suitable for the intended administration. Suitable forms of the pre- or prodrugm or functionally active protein produced as an active pharmaceutical ingredient, through recombinant DNA technology, include pharmaceutically (i.e. physiologically) acceptable salts, formulations, and excipients, known to those skilled in the art, for the compound(s) of the invention.

As used herein, “effective” as in an amount effective to achieve an end means the quantity of a component that is sufficient to yield an indicated therapeutic response without undue adverse side effects (such as toxicity, irritation, or allergic response) commensurate with a reasonable benefit/risk ratio when used in the manner of this disclosure. For example, an amount effective to treat a fibroproliferative disorder. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

As used herein, an “amount” of a compound as measured in milligrams refers to the milligrams of compound present in a preparation, regardless of the form of the preparation. An “amount of compound which is 90 mg” means the amount of the compound in a preparation is 90 mg, regardless of the form of the preparation. Thus, when in the form with a carrier, the weight of the carrier necessary to provide a dose of 90 mg compound would be greater than 90 mg due to the presence of the carrier.

As used herein, “about” in the context of a numerical value or range means±10% of the numerical value or range recited or claimed.

As used herein, to “treat” or “treating” encompasses, e.g., inducing inhibition, regression, or stasis of the disorder and/or disease. As used herein, “inhibition” of disease progression or disease complication in a subject means preventing or reducing or reversing the disease progression and/or disease complication in the subject.

The human matrix metalloproteinase is a collagenase.

As used herein, human matrix metalloproteinase refers to both natural forms, forms produced by recombinant DNA technology, which is understood by a person with ordinary skill in the art, and mutated forms having analogous activity. For example, human matrix metalloproteinase can be as found in nature as in Gross, 1962, or it can be in mutated form as in Paladini, 2013, the contents of which are hereby incorporated by reference in its entirety. Human matrix metalloproteinase produced by DNA technology can be made using prokaryotic or eukaryotic cells or other host systems known to those skilled in the art of protein expression, that yield functional enzyme.

Further, as described in Paladini, 2013, it is possible to create a mutated human matrix metalloproteinase, such as mutated MMP-1, wherein the activity can be modulated by the concentration of Ca2+. This can give the ability to control the in vivo activity of the mutated human matrix metalloproteinase, such as mutated MMP-1

Any known TNF antagonist may be utilized in the implementation of the invention, a broad variety of which are known and disclosed in the art. The TNF antagonist is preferably a human TNF-α antagonist. Optionally, the TNF antagonist may be an antibody, such as a monoclonal antibody or fragment thereof; a chimeric monoclonal antibody (such as a human-murine chimeric monoclonal antibody); a fully human monoclonal antibody; a recombinant human monoclonal antibody; a humanized antibody fragment; a soluble TNF antagonist, including small molecule TNF blocking agents such as thalidomide or analogues thereof or PDE-IV inhibitors; a TNF receptor or a TNF receptor fusion protein, e.g. a soluble or TNF receptor or TNF receptor fusion protein. Optionally, the TNF antagonist is a functional fragment or fusion protein comprising a functional fragment of a monoclonal antibody, e.g. of the 15 types mentioned above, such as a Fab, F(ab′)2, Fv and preferably Fab. Preferably a fragment is pegylated or encapsulated (e.g. for stability and/or sustained release).

Optionally, the TNF antagonist is provided as a bi-functional (or bi-specific) antibody or bi-functional (or bi-specific) antibody fragment. The bifunctional TNF-α antagonist antibody or fragment thereof may be, for example, an antibody, such as a monoclonal antibody or fragment thereof, a chimeric monoclonal antibody (such as a human-murine chimeric monoclonal antibody), a fully human monoclonal antibody, a recombinant human monoclonal antibody, a humanized antibody fragment. Where the TNF-α antagonist comprises a bifunctional antibody fragment or portion, it is preferably a bi-functional F(ab′)2 fragment or divalent ScFv, e.g. a bi-specific tandem di-ScFv. In any case, the bifunctional (or bi-specific) antibody or fragment thereof may comprise as one variable domain (e.g. antigen binding portion) a TNF-α antagonist (e.g. a TNF-α antagonist portion of Infliximab, Adalimumab, Certolizumab, Golimumab, pegol or Etanercept) and as the other variable domain (e.g. antigen binding portion) a 30 second variable domain other than TNF-α antagonist. Optionally, the second variable domain may comprise an antibody mobility inhibitor, which may be, for example an extracellular matrix, e.g. collagen, binder or antagonist. Thereby, a higher dose of TNF-α antagonist may be administered since the antibody or fragment thereof will be self-Iocalising, minimizing systemic uptake and thus systemic side effects. Optionally, the second variable domain may comprise a DAMP antagonist (such as an antagonist for S100A8 and/or S100A9, e.g. as described in U.S. Pat. No. 7,553,488) or an AGE inhibitor (e.g. being variable domains of DAMP antagonist antibody or AGE inhibitor antibody). Methods for the production of bifunctional antibodies, and bi-functional antibody fragments are known in the art, which methods may be applied to the present purpose.

Preferably, the TNF-α antagonist is selected from those which at administration (e.g. local administration, such as injection into clinical nodule or cord) cause administration-site irritation manifested as palpable local swelling, redness and pruritis in fewer than 40% of patients, preferably fewer than 20% and more preferably fewer than 10%.

The TNF-α antagonist may be selected, for example, from one or a combination of Infliximab, Adalimumab, Certolizumab pegol, Golimumab or Etanercept, or functional fragment thereof. Most preferably, the TNF-α antagonist is Certolizumab pegol, since it causes low injection site reaction and pain.

Other TNF antagonists are disclosed in Tracey, 2008, the contents of which are hereby incorporated by reference.

By early disease state it is meant that indications of disease are present, e.g. histological markers or more particularly clinical nodules in tissue, but in the absence of, for example, palpable cord or significant contracture. By early disease state Dupuytren's disease, it is meant that indications of Dupuytren's disease are present, e.g. histological markers or more particularly clinical nodules 20 in palmar and/or digital tissue, but in the absence of significant (e.g. at least 5°) flexion contracture (or, for example, palpable cord).

By established disease state, it is meant that clinical nodules are present, palpable cord is present and contracture is evident. By established disease state Dupuytren's disease, it is meant that clinical nodules are present on the palm 25 and digits of the hand and flexion contracture is evident (e.g. at least 5°).

Varying histological stages of Dupuytren's disease have been categorised in the literature, most succinctly by Rombouts, 1989 and later authors, into three distinct stages: 1) a proliferative stage with high cellularity and the presence of mitotic figures; 2) a fibrocellular stage charactised by high cellularity but no mitotic figures and the presence of reticulin network; and 3) a fibrous stage with few cells separated by broad bundles of collagen fibres. Stage 1) disease is believed to correlate with early disease state as discussed above (i.e. presence of nodules but no contracture) and Dupuytren's stages 2) and 3) is believed to correlate with our Established Disease State (characterized by digital contracture). The present inventors have found that during early established disease state, active myofibroblasts are collected in the established nodules and cords, especially in relation to the MCP and PIP joints and these drive the progression of flexion contractures of the digit.

In certain claims, the invention claims the amount of the TNF antagonist as a multiple of the clinical dose administered for Rheumatoid Arthritis. For example, if a claim states 0.1 to 3.0 times the clinical dose administered for Rheumatoid Arthritis, and the clinical dose administered for Rheumatoid Arthritis for that particulate TNF antigen is 100 mg, then the dose of the TNF antagonist for the claimed method is between 10 mg and 300 mg. This example is for understanding only and does not limit the invention in any way.

The following chart identifies and briefly describes the different human matrix metalloproteinase.

| Gene | Name | Location | Description |
|---|---|---|---|
| MMP1 | Interstitial | secreted | Substrates include Col I, II, III, |
| | collagenase | | VII, VIII, X, gelatin |
| MMP2 | Gelatinase-A, | secreted | Substrates include Gelatin, Col I, |
| | 72 kDa | | II, III, IV, Vii, X |
| | gelatinase | | |
| MMP3 | Stromelysin 1 | secreted | Substrates include Col II, IV, IX, |
| | | | X, XI, gelatin< |
| MMP7 | Matrilysin, | secreted | membrane associated through |
| | PUMP 1 | | binding to cholesterol sulfate in |
| | | | cell membranes, substrates |
| | | | include: fibronectin, laminin, Col |
| | | | IV, gelatin |
| MMP8 | Neutrophil | secreted | Substrates include Col I, II, III, |
| | collagenase | | Vii, VIII, X, aggrecan, gelatin |
| MMP9 | Gelatinase-B, | secreted | Substrates include Gelatin, Col |
| | 92 kDa | | IV, V |
| | gelatinase | | |
| MMP10 | Stromelysin 2 | secreted | Substrates include Col IV, |
| | | | laminin, fibronectin, elastin |
| MMP11 | Stromelysin 3 | secreted | MMP-11 shows more similarity to |
| | | | the MT-MMPs, is convertase- |
| | | | activatable and is secreted |
| | | | therefore usually associated to |
| | | | convertase-activatable MMPs. |
| | | | Substrates include Col IV, |
| | | | fibronectin, laminin, aggrecan |
| MMP12 | Macrophage | secreted | Substrates include Elasin, |
| | metalloelastase | | fibronectin, Col IV |
| MMP13 | Collagenase 3 | secreted | Substrates include Col I, II, III, |
| | | | IV, IX, X, XIV, gelatin |

The following chart summarizes the treatments to patients.

| | | | | | | | Collagenase | | |
|---|---|---|---|---|---|---|---|---|---|
| | | Intralesional | | | Preferred | | Preferred | | Anti-TNF |
| | | Injection - into | | Time between | Time between | Collagenase | dose range | Anti-TNF | Preferred |
| | intravenous | disease issue | Injection order | injections | injections | dose range | (mg) | dose range | dose range |
| collagenase | | + | 1st | 0 min to 48 hrs | | 0.01-10 mg | 0.1-2.0 | | |
| Anti-TNF | | + | 2nd | Inject | 1) 24 hrs | | | 0.05-5.0X | 0.1-3X |
| | | | | within 48 | or | | | Clinical | clinical |
| | | | | hour post | 2) 1-30 min | | | dose | dose for |
| | | | | collagenase | | | | for | rheumatoid |
| | | | | | | | | R.A. | arthritis |
| collagenase | | + | 2nd | Inject | 1) 24 hrs | 0.01-10 mg | 0.1-2.0 | | |
| | | | | within 48 | or | | | | |
| | | | | hour post | 2) 1-60 min | | | | |
| | | | | anti-TNF | | | | | |
| Anti-TNF | | + | 1st | | | | | 0.05-5.0X | 0.1-3X |
| | | | | | | | | Clinical | clinical |
| | | | | | | | | dose | dose for |
| | | | | | | | | for | rheumatoid |
| | | | | | | | | R.A. | arthritis |
| collagenase | | + | 2nd or | 0 min to 48 hrs | 1) 24 hrs | 0.01-10 mg | 0.1-2.0 | | |
| | | | 1st | | or | | | | |
| | | | | | 2) 1-60 min | | | | |
| Anti-TNF | + | | 1st or | Inject | 1) 24 hrs | | | 0.05-5.0X | 0.1-3X |
| | | | 2nd | within 48 | or | | | Clinical | clinical |
| | | | | hour post | 2) 1-60 min | | | dose | dose for |
| | | | | collagenase | | | | for | rheumatoid |
| | | | | | | | | R.A. | arthritis |

### REFERENCES CITED

Hurst et al. “Injectable Collagenase Clostridium Histolyticum for Dupuytren's Contracture” N ENGL J MED 361; 10 Sep. 3, 2009
Paladini et al. “Mutations in the Catalytic Domain of Human Matrix
Metalloproteinase-1 (MMP-1) That Allow for Regulated Activity through the Use of Ca2+” THE JOURNAL OF BIOLOGICAL CHEMISTRY VOL. 288, NO. 09. pp. 6629-6639, Mar. 1, 2013
Verjee at al., “Unraveling the signaling pathways promoting fibrosis
in Dupuytren's disease reveals TNF as a therapeutic target” PNAS vol. 110 no. 10 published Feb. 19, 2013.
Kontermann, Roland E. “Dual targeting strategies with bispecific antibodies” mAbs 4:2, 182-197; March/April 2012
Tracy et al. “Tumor necrosis factor antagonist mechanisms of action: A comprehensive review” Pharmacology & Therapeutics 117 (2008) 244-279
Summary basis of approval XIAFLEX-EU: Available at www.ema.europa.eu/docs/en_GB/document_library/EPAR-_Product_Information/human/002048/WC500103373.pdf
Rombouts, J Hand Surg Am, 14, 644-652, 1989

1. A method of treating a subject afflicted with a fibroproliferative disorder comprising periodically administering to the patient an amount of one or more human matrix metalloproteinase, wherein the one or more human matrix metalloproteinase is or are selected from the group consisting of human metalloproteinase-1 (MMP-1), human metalloproteinase-2 (MMP-2), human metalloproteinase-3 (MMP-3), human metalloproteinase-7 (MMP-7), human metalloproteinase-8 (MMP-8), human metalloproteinase-9 (MMP-9), human metalloproteinase-10 (MMP-10), human metalloproteinase-11 (MMP-11), metalloproteinase-12 (MMP-12), and human metalloproteinase-13 (MMP-13), and wherein the amount is effective to treat the subject.

2. The method of claim 1 further comprising periodically administering to the subject an amount of a TNF antagonist, wherein the amount of said one or more the human matrix metalloproteinase and the amount of said TNF antagonist when taken together are effective to treat the subject.

3. The method of claim 2 wherein said one or more human matrix metalloproteinase and said TNF antagonist are administered adjunctively or concomitantly.

4. The method of claim 2, wherein the fibroproliferative disorder is a fibromatosis disease.

5. The method of claim 2, wherein the fibroproliferative disorder is selected from the group consisting of Dupuytren's disease, plantar fibromatosis, adhesive capsulitis and Peyronie's disease.

6. The method of claim 2, wherein the fibroproliferative disorder is Dupuytren's disease.

7. The method of claim 2, wherein said one or more human matrix metalloproteinase is human metalloproteinase-1 (MMP-1).

8. The method of claim 2, wherein the fibroproliferative disorder is an early disease state fibroproliferative disorder.

9. (canceled)

10. The method of claim 2, wherein said one or more human matrix metalloproteinase and said TNF antagonist are administered directly into diseased tissue.

11. The method of claim 2, wherein the amount of one or more human matrix metalloproteinase and/or the amount of TNF antagonist are administered topically onto disease tissue or intravenously.

12. (canceled)

13. The method of claim 2, wherein the TNF antagonist is Infliximab, Adalimumab, Certolizumab pegol, Golimumab or Etanercept.

14. The method of claim 2, wherein a therapeutic, prophylactic or progression-inhibiting amount of a DAMP antagonist, an AGE inhibitor, or both is or are also administered to the subject.

15. The method of claim 2, wherein a therapeutic, prophylactic or progression-inhibiting amount of an Alarmin antagonist or an AGE inhibitor or both is or are also administered to the subject.

16. The method of claim 15, wherein said Alarmin antagonist is HMGB1, S100A8, S100A9, SI00A8/9 or S100A12.

17. The method of claim 2, wherein said one or more human matrix metalloproteinase and TNF antagonist administered daily, weekly, biweekly, monthly or bimonthly.

18. (canceled)

19. The method of claim 2, wherein the amount of said one or more human matrix metalloproteinase administered is between 0.01 and 10 mg.

20-49. (canceled)

50. A kit for use in treating a subject afflicted with a fibroproliferative disorder comprising:
i. a syringe or vial containing one or more human matrix metalloproteinase wherein the one or more human matrix metalloproteinase is selected from human metalloproteinase-1 (MMP-1), human metalloproteinase-2 (MMP-2), human metalloproteinase-3 (MMP-3), human metalloproteinase-7 (MMP-7), human metalloproteinase-8 (MMP-8), human metalloproteinase-9 (MMP-9), human metalloproteinase-10 (MMP-10), human metalloproteinase-11 (MMP-11), metalloproteinase-12 (MMP-12), and human metalloproteinase-13 (MMP-13); and/or
ii. a syringe or vial containing a TNF antagonist

51. A method of treating a human patient afflicted with a fibroproliferative disorder comprising periodically administering to the patient an amount of a bi-specific antibody comprising a human matrix metalloproteinase specificity and a TNF antagonist specificity.

52-54. (canceled)
